(19) United States
(12) Patent Application Publication
(10) Pub. No.: US 2023/0377320 A1
(43) Pub. Date: Nov. 23, 2023

(54) DEEP LEARNING MODEL TRAINING OF X-RAY AND CT

(71) Applicant: KONINKLIJKE PHILIPS N.V., EINDHOVEN (NL)

(72) Inventors: Xin WANG, BELMONT, MA (US); Sandeep Madhukar DALAL, WINCHESTER, MA (US); Saifeng LIU, CAMBRIDGE, MA (US)

(21) Appl. No.: 18/031,017

(22) PCT Filed: Sep. 30, 2021
(86) PCT No.: PCT/EP2021/076911

§ 371 Date: Apr. 10, 2023

Related U.S. Application Data
(60) Provisional application No. 63/090,745, filed on Oct. 13, 2020.

(51) Int. Cl. G06V 10/80; G06V 10/774; G06T 7/00

(52) U.S. Cl. CPC G06V 10/811; G06V 10/774; G06T 7/0012; G06T 2207/10081; G06T 2207/10116; G06T 2207/20081; G06V 2201/03

(57) ABSTRACT

A system and method for training a deep learning network with images of a first modality and images of a second modality to predict a diagnosis for a current image study of one of the first and second modalities. The training includes collecting training data including a plurality of datasets, each dataset including an image study of the first modality and an image study of the second modality for a single patient and clinical reason, training a first branch of the deep learning network with images of the first modality and training a second branch of the deep learning network with images of the second modality.

### BACKGROUND

X-ray imaging is often used to diagnose injuries and/or diseases as it is one of the most cost-effective medical imaging examinations and is easily accessible. For example, X-ray images may be used to detect dislocations and fractures of bone, cancers, and lung or chest problems. Determining a diagnosis based on X-ray images, however, is generally regarded as more challenging than determining a diagnosis via, for example, CT (Computed Tomography) imaging. CT scans combine a series of X-ray images taken from many different angles to produce cross-sectional images which, together, provide a three-dimensional image of a target portion of a patient's body. An X-ray provides a two-dimensional image of the target portion of the patient's body and therefore does not present as much information as a CT scan. In some cases, use of X-ray images may fail to diagnose problems with, for example, muscle damage, soft tissues or other body organs. In one example, while most bone fractures are readily discernible via CT, some fractures may be missed if imaged via an X-ray alone. This is especially common with, for example, wrist fractures, hip fractures, and stress fractures, so that an additional imaging examination (e.g., CT, MRI or bone scan) may be required. Imaging examinations such as CT, however, are typically more costly than X-rays and may not be readily available.

Automated diagnostic systems utilizing, for example, machine learning, have been playing an increasingly important role in healthcare. Currently, deep learning models for detecting findings based on X-ray images have been developed. These deep learning models are only trained using X-ray images and are thus unable to apply knowledge from cases in which that patient required more than one image study—e.g., an X-ray and a CT scan—to confirm a diagnosis.

### SUMMARY

Some exemplary embodiments are related to a computer-implemented method of training a deep learning network with images of a first modality and images of a second modality to predict a diagnosis for a current image study of one of the first and second modalities. The method includes collecting training data including a plurality of datasets, each dataset including an image study of the first modality and an image study of the second modality for a single patient and clinical reason, training a first branch of the deep learning network with images of the first modality and training a second branch of the deep learning network with images of the second modality.

Other exemplary embodiments are related to a system of training a deep learning network with images of a first modality and images of a second modality to predict a diagnosis for a current image study of one of the first and second modalities. The system includes a non-transitory computer readable storage medium storing an executable program and a processor executing the executable program. The program causes the processor to collect training data including a plurality of datasets, each dataset including an image study of the first modality and an image study of the second modality for a single patient and clinical reason, train a first branch of the deep learning network with images of the first modality and train a second branch of the deep learning network with images of the second modality.

Still further exemplary embodiments are related to a non-transitory computer-readable storage medium including a set of instructions executable by a processor. The set of instructions, when executed by the processor, cause the processor to perform operations. The operations include collecting training data including a plurality of datasets, each dataset including an image study of the first modality and an image study of the second modality for a single patient and clinical reason, training a first branch of the deep learning network with images of the first modality and training a second branch of the deep learning network with images of the second modality.

### BRIEF DESCRIPTION OF THE DRAWINGS

FIG. 1 shows a schematic diagram of a system according to an exemplary embodiment.

FIG. 2 shows a schematic diagram of a deep learning model architecture of the system according to FIG. 1.

FIG. 3 shows a flow diagram of a method for deep learning of both X-ray and CT images according to an exemplary embodiment.

### DETAILED DESCRIPTION

The exemplary embodiments may be further understood with reference to the following description and the appended drawings, wherein like elements are referred to with the same reference numerals. The exemplary embodiments relate to systems and methods for machine learning and, in particular, relate to systems and methods for training a neural network of a deep learning model with both X-ray images and CT images to enhance diagnostic and/or predictive capabilities of the deep learning model. Training data may include both X-ray and CT images that have been acquired for the same patient and for the same clinical reason and/or during the same period of time. Thus, an accuracy of the X-ray model is improved via matched CT images so that, during an inference stage, the deep learning model may be applied to just an X-ray image to interpret the image and/or determine a diagnosis. It will be understood by those of skill in the art that although the exemplary embodiments are shown and described with respect to X-rays and CT scans, the systems and methods of the present disclosure may be similarly applied to any of a variety of medical imaging modalities in any of a variety of medical fields for any of a variety of different pathologies.

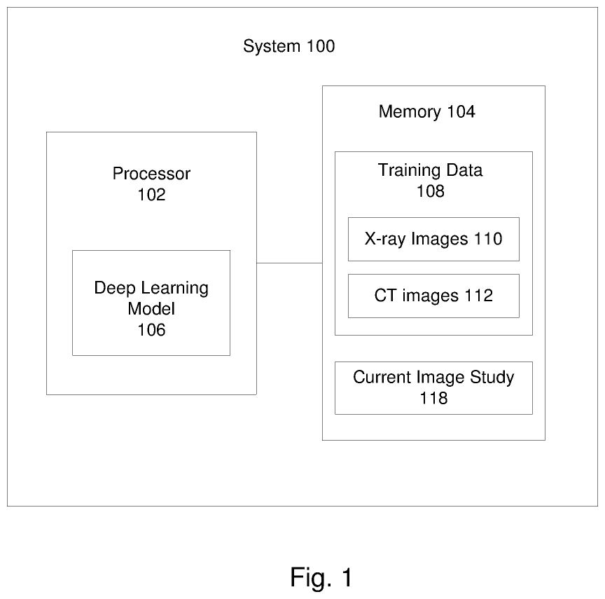

As shown in FIG. 1, a system 100 according to an exemplary embodiment of the present disclosure trains a neural network of a deep learning model 106 with training data 108 including both images of a first modality and images of a second modality to provide a diagnosis based on an image of one of the first and second modalities. The system 100 comprises a processor 102 including or executing the deep learning model 106. In one embodiment, the deep learning model 106 is trained using training data 108 including datasets, each dataset including an X-ray image 110 and a corresponding CT image 112. The X-ray image 110 and the corresponding CT image 112 of each of the datasets are acquired from the same patient for the same clinical reason and/or within the same period of time. Such data is likely to be available for patients who get both X-ray and CT exams ordered when they visit, for example, an emergency department to diagnose their condition.

The processor 102 may be configured to execute computer-executable instructions for operations from applications that provide functionalities to the system 100, including instructions for training of the deep learning model 106. It should be noted, however, that functionalities described with respect to the deep learning model 106 may also be represented as a separately incorporated component of the system 100, a modular component connected to the processor 102 or as a functionalities achievable via more than one processor 102. For example, the system 100 may be comprised of a network of computing systems, each of which includes one or more of the components described above. It will be understood by those of skill in the art that although the system 100 shows and describes a single deep learning model 106, the system 100 may include a plurality of deep learning models 106, each learning model trained with training data corresponding to a different target portion of the patient body and/or a different pathology.

Although the exemplary embodiments show and describe the training data 108 as being stored to the memory 104, it will be understood by those of skill in the art that datasets of the training data 108 may be acquired from any of a plurality of databases stored by any of a plurality of devices connected to and accessible via the system 100 via, for example, a network connection. In one exemplary embodiment, the training data 108 may be acquired from one or more remote and/or network memories and stored to a central memory 104. Alternatively, the training data may be collected and stored to any remote and/or networked memory.

Similarly, a current image study 118 to be interpreted via the trained deep learning model 106 may be acquired and received from any imaging device. It will be understood by those of skill in the art that the imaging device may transmit the current image study 118 to the system 100 and/or be in network with the system 100. The current image study 118 may similarly be received via the processor 102 and/or stored to the memory 104 or any other memory, remote or in network. The current image study 118 may have any of a variety of modalities and in one particular embodiment, includes an X-ray so that the current image study may be interpreted based on deep learning of the X-ray images 114 of the training data 108, which are enhanced via the matched CT images. Although the system 100 shows a single current image study 118, it will be understood by those of skill in the art that the system 100 may include more than one current image study for the same patient for the same clinical reason. In one example, the system 100 may receive both an X-ray image and a CT image to be interpreted via the deep learning model 106.

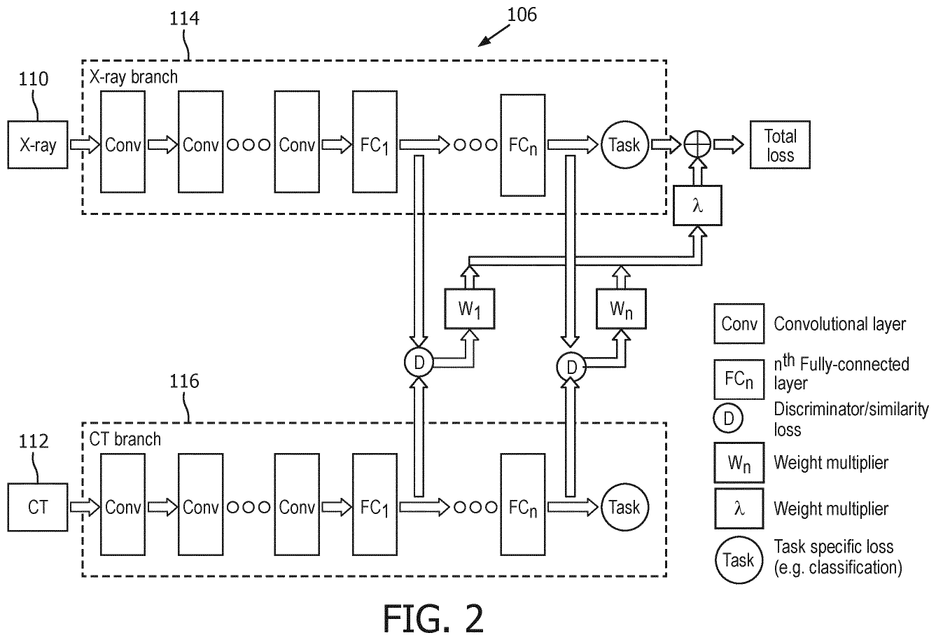

As shown in FIG. 2, in one embodiment, the deep learning model 106 may include a neural network including two branches—a first X-ray branch 114 trained via the X-ray images 110 and a second CT branch 116 trained via the CT images 112. Each of the branches 114, 116 include a plurality of convolutional layers, from which feature maps are converted to a feature vector. Following the convolutional layers, the branches 114, 116 include a plurality of fully connected layers. It will be understood by those of skill in the art that the convolutional layers will vary between the first and second branches 114, 116. The first and second branches 114, 116, however, will share the same architecture from a first one of the fully connected layers to a final, fully connected output layer.

As will be described in further detail below, in one embodiment, the processor first trains the CT branch 116 with the CT images 110 of the training data 108. Upon completion of training of the CT branch 116, weights may be frozen and may not be trained during training of the X-ray branch 114. The processor 102 may calculate similarity losses from different pairs of feature vectors—e.g., X-ray vs. CT—and combine the similarity losses through weighted averaging. These similarity losses along with classification losses of the CT branch may be used to determine classification loss of the X-ray branch. A weighted summation of the X-ray classification loss is optimized through cross validation and/or learned during training of the deep learning model 106.

Where, for example, the current image study 118 to be interpreted is an X-ray, upon completion of training of the deep learning model 106, during an inference stage, the X-ray branch 114 of the deep learning model 106 may be applied to determine a diagnostic prediction for the current image study 118. Where the system 100 receives both an X-ray and a CT to be interpreted for the same person and for the same clinical reason, both the X-ray branch 114 and the CT branch 116 may be applied to determine a prediction.

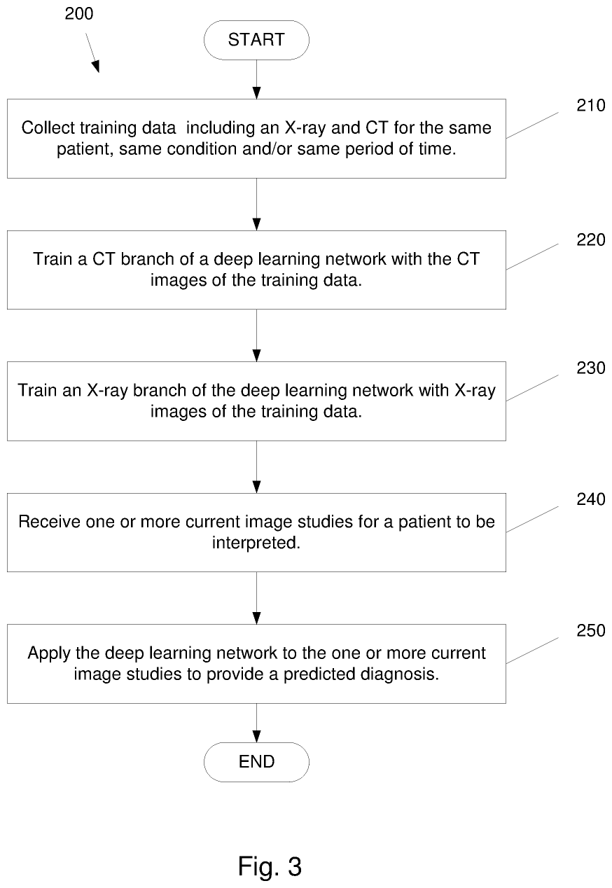

FIG. 3 shows an exemplary method 200 for the deep learning model 106 of the system 100. As described above, the deep learning model 106 is capable of providing a diagnosis and/or prediction of disease or injury for an X-ray image based on learned data from X-ray images and their corresponding CT images. In 210, training data 108 comprising data sets including, for example, X-ray images 114 and corresponding CT images 116 are collected. The training data 108 may be collected and stored to the memory 108. In particular, each dataset includes X-ray and CT image examinations acquired for the same patient and for the same clinical reason and/or during the same period of time. A patient's longitudinal record may be helpful for identifying X-ray and CT images which were performed for the purpose of diagnosing the same condition.

In 220, the CT branch 116 of the deep learning model 106 is trained using CT images 112 collected as part of the training data 108. The CT branch 116 learns the CT images 112 via a plurality convolutional layers applying filters to each of the CT images 116 until a feature map for each CT image 112 is derived. The feature maps are then converted to a feature vector of the same size, which is followed by a plurality of fully connected layers representative of each of the feature vectors of the feature map. Upon completion of training of the CT branch 116 using the CT images 112, weights of the CT branch are frozen in 230. Loss for the CT branch may be calculated as the classification loss shown in the equation below.

Loss_{_CT}=Loss_{_cl_CT}

In 230, the X-ray branch 114 is trained using the X-ray images from the training data 106. Since the CT branch 116 is frozen, the CT branch 116 will not be retrained during the training of the X-ray branch 114. Similar to the CT branch 116, the X-ray branch 114 learns the X-ray images 114 via a plurality convolutional layers applying filters to each of the X-ray images 114 until a feature map for each X-ray image 110 is derived. The feature maps are then converted to a feature vector of the same size, which is followed by a plurality of fully connected layers for the feature vectors. As described above, an architecture of the X-ray branch 114 and the CT branch 116 varies for the convolutional layers. The X-ray branch 114 and the CT branch 116, however, will share the same architecture for the fully connected layers.

For the training of the X-ray branch 114, a similarity metric such as L2 norm, L1 norm, hybrid norm (e.g., Huber), Cosine similarity, Wasserstein distance or a pretrained discriminator network may be used to evaluate the similarity between the feature vectors of the two branches—e.g., the feature vectors of the X-ray branch 114 and the feature vectors of the CT branch 116. Feature vectors will be normalized before the calculation of the similarity metric. A negative value of this similarity is defined as the similarity loss between the X-ray branch 114 and the CT branch 116.

Loss_{_similarity}=−Similarity(feature vectors from CT branch, feature vectors from X-ray branch)

The similarity losses obtained from different pairs of feature vectors (X-ray vs. CT) is combined through weighted averaging. The weights for combining the similarity losses are to be learned during training. The final loss function of the X-ray branch is defined as another weighted summation of the X-ray classification loss and the similarity loss as in the equation below.

Loss_{_Xray}=Loss_{_cl_Xray}+λ*Loss_{_similarity}

The weight λ may be optimized through cross validation and/or learned during training. Thus, during the training stage, the method 200 minimizes classification loss in the X-ray branch 114 while also minimizing its distance from feature vectors in the CT branch 116.

Although training of the CT branch 116 and the X-ray branch 114 are shown and described above as being individually trained in 230 and 240, respectively, in another embodiment, the X-ray branch 114 and CT branch 116 may be trained simultaneously by using a loss function that is defined as a weighted summation of the CT classification loss, the X-ray classification loss and the similarity loss as in the equation below.

Loss_{_comb}=Loss_{_cl_CT}+α*Loss_{_cl_Xray}+λ*Loss_{_similarity}

The weights α and λ may be optimized through cross validation and/or learned during training. Thus, during the training stage, the method 200 minimizes both classification loss in the CT branch 116 and classification loss in the X-ray branch 114 while also minimizing the distance between feature vectors in the CT branch 116 and the feature vectors in the X-ray branch 114.

Upon completion of training of the deep learning module, the method 200 may proceed to an inference stage in which a current image study 118 is to be interpreted. In 240, the processor 102 receives the current image study 118 to be interpreted. The current image study 118 includes one of the image modalities used to train the deep learning model 106. In one embodiment, the current image study 118 may include an X-ray image. It will be understood by those of skill in the art, however, that where the deep learning model is trained using both an X-ray and CT images, the system 100 may receive one or more current image studies 118, which may include both an X-ray image and a CT image acquired for the same patient and for the same clinical reason.

In 250, the deep learning model 106 is applied to the current image study 118 to provide a predictive diagnosis based on the current image study. Where the current image study is an X-ray, the predictive diagnosis is based on the X-ray branch 114 of the deep learning model, which is enhanced with knowledge from the corresponding CT images from the CT branch 116. Where more than one current image study 118 (e.g., X-ray and CT) is to be interpreted, both the X-ray branch 114 and the CT branch 116 may be applied, improving an accuracy of the predictive diagnosis.

Those skilled in the art will understand that the above-described exemplary embodiments may be implemented in any number of manners, including, as a separate software module, as a combination of hardware and software, etc. For example, the deep learning model 106 may be a program including lines of code that, when compiled, may be executed on the processor 102.

Although this application described various embodiments each having different features in various combinations, those skilled in the art will understand that any of the features of one embodiment may be combined with the features of the other embodiments in any manner not specifically disclaimed or which is not functionally or logically inconsistent with the operation of the device or the stated functions of the disclosed embodiments.

It will be apparent to those skilled in the art that various modifications may be made to the disclosed exemplary embodiments and methods and alternatives without departing from the spirit or scope of the disclosure. Thus, it is intended that the present disclosure cover the modifications and variations provided that they come within the scope of the appended claims and their equivalents.

1. A computer-implemented method of training a deep learning network with images of a first modality and images of a second modality to predict a diagnosis for a current image study of one of the first and second modalities, comprising:
collecting training data including a plurality of datasets, each dataset including an image study of the first modality and an image study of the second modality for a single patient and clinical reason;
training a first branch of the deep learning network with images of the first modality; and
training a second branch of the deep learning network with images of the second modality,
wherein the training of the second branch is enhanced by the training of the first branch.

2. The method of claim 1, further comprising:
receiving a current image study to be interpreted; and
applying the deep learning network to the current image study to interpret the current image study.

3. The method of claim 1, wherein training the first branch of the deep learning network includes, for each image of the first modality, a plurality of convolutional layers deriving a feature map of each image of the first modality, and a plurality of fully connected layers for feature vectors of the feature map of each image of the first modality.

4. The method of claim 3, wherein training the second branch of the deep learning network includes, for each image of the second modality, a plurality of convolutional layers deriving a feature map of each image of the second modality, and a plurality of fully connected layers for feature vectors of the feature map of each image of the second modality.

5. The method of claim 4, further comprising combining similarity losses obtained from pairs of featured vectors of the first image modality and the second image modality through weighted averaging.

6. The method of claim 5, wherein a final loss of the second branch of the deep learning network is defined via a classification loss of the second branch of the deep learning network and the combined similarity losses obtained from the pairs of feature vectors.

7. The method of claim 1, further comprising, subsequent to training the first branch of the deep learning network and prior to training of the second branch of the deep learning network, freezing the first branch of the deep learning network so that the first branch is not retrained while the second branch is being trained.

8. The method of claim 1, wherein the first branch and the second branch are trained simultaneously so that a loss function is defined.

9. The method of claim 1, wherein the first and second image modalities include a CT and an X-ray.

10. A system of training a deep learning network with images of a first modality and images of a second modality to predict a diagnosis for a current image study of one of the first and second modalities, comprising:
a non-transitory computer readable storage medium storing an executable program; and
a processor executing the executable program to cause the processor to:
collect training data including a plurality of datasets, each dataset including an
image study of the first modality and an image study of the second modality for a single
patient and clinical reason;
train a first branch of the deep learning network with images of the first modality;
and
train a second branch of the deep learning network with images of the second
modality,
wherein training of the second branch is enhanced by knowledge from the first branch.

11. The system of claim 10, wherein the processor executes the executable program to cause the processor to:
receive a current image study to be interpreted; and
apply the deep learning network to the current image study to interpret the current image study.

12. The system of claim 10, wherein the first branch of the deep learning network includes, for each image of the first modality, a plurality of convolutional layers deriving a feature map of each image of the first modality, and a plurality of fully connected layers for feature vectors of the feature map of each image of the first modality.

13. The system of claim 12, wherein the second branch of the deep learning network includes, for each image of the second modality, a plurality of convolutional layers deriving a feature map of each image of the second modality, and a plurality of fully connected layers for feature vectors of the feature map of each image of the second modality.

14. The system of claim 13, wherein the processor executes the executable program to cause the processor to combine similarity losses obtained from pairs of featured vectors of the first image modality and the second image modality through weighted averaging.

15. The system of claim 14, wherein the processor executes the executable program to cause the processor to define a final loss of the second branch of the deep learning network via a classification loss of the second branch of the deep learning network and the combined similarity losses obtained from the pairs of feature vectors.

16. The system of claim 10, wherein the processor executes the executable program to cause the processor to freeze the first branch of the deep learning network so that the first branch is not retrained while the second branch is being trained.

17. The system of claim 10, further comprising a memory storing the training data including the plurality of datasets.

18. The system of claim 10, wherein the first branch and the second branch are trained simultaneously so that a loss function is defined as a weighted summation of a classification loss of the first branch, a classification loss of the second branch and a combined loss similarity obtained from pairs of the first and second image modalities.

19. The system of claim 18, wherein the first and second image modalities include a CT and an X-ray.

20. A non-transitory computer-readable storage medium including a set of instructions executable by a processor, the set of instructions, when executed by the processor, causing the processor to perform operations, comprising:
collecting training data including a plurality of datasets, each dataset including an image study of the first modality and an image study of the second modality for a single patient and clinical reason;
training a first branch of the deep learning network with images of the first modality; and
training a second branch of the deep learning network with images of the second modality and knowledge from the first branch.
